(19) United States
(12) Patent Application Publication
(10) Pub. No.: US 2022/0039784 A1
(43) Pub. Date: Feb. 10, 2022

(54) REMOTELY POWERED ARTICULATED MEDICAL APPARATUS

(71) Applicant: CANON U.S.A., INC., Melville, NY (US)

(72) Inventors: Charles George Hwang, Wellesley, MA (US); Matthew Michael Kincaid, Medford, MA (US); Brian Ninni, Brighton, MA (US)

(21) Appl. No.: 17/396,274

(22) Filed: Aug. 6, 2021

Related U.S. Application Data
(60) Provisional application No. 63/062,235, filed on Aug. 6, 2020.

(51) Int. Cl. A61B 17/00; A61B 10/04; A61B 1/00

(52) U.S. Cl. CPC A61B 17/00234; A61B 10/04; A61B 2017/00544; A61B 2017/00327; A61B 2017/00539; A61B 1/00154

(57) ABSTRACT

One or more apparatus and systems for articulated movement, particularly for an articulated medical device such as an endoscope or medical robot. The apparatus comprises an articulated movement portion with tendon control, a transition portion comprising actuation rods, springs, and distal stoppers, a conduit portion comprising lumen filled with air or hydraulic fluid; a translation portion comprising a proximal stopper; and a power unit.

### CROSS REFERENCE TO RELATED APPLICATIONS

The present application claims the benefit of priority from U.S. Provisional Patent Application No. 63/062,235 filed Aug. 6, 2020. The disclosure of the above-listed provisional application is hereby incorporated by reference in its entirety for all purposes. Priority benefit is claimed under 35 U.S.C. § 119(e).

### BACKGROUND

### Field of the Disclosure

The present disclosure relates generally to the medical robotics and particularly catheters and endoscopes and to the field of hydraulic/pneumatic lines.

### Background

There are various catheters and endoscopes that have been developed to steer a medical device to a target for visualization and/or surgery. Many provide a simple bend, but some are steerable and have tendons that can push the end or other portions of the device to steer the catheter in a particular direction. Tendons with both push and pull functionality are also described. However, directly actuating articulating wires/cables over a distance can be cost, size, force, and weight prohibitive, particularly in medical devices where a small size can be necessary.

Hydraulic/pneumatic lines to apply force at a distance, none have been used to push/pull structures which, in turn, cause articulated movement. For example, U.S. Pat. No. 5,464,394 which describe hydraulically applying force in conjunction with a return spring to move an object.

Thus, it would be advantageous to provide a catheter or endoscopy using hydraulic/pneumatic lines to actuate movement.

### SUMMARY OF THE DISCLOSURE

Accordingly, it is a broad object of the present disclosure to provide a medical apparatus comprising an articulated movement portion. This portion comprises a central lumen extending the length of the articulated movement portion, a wall formed about the central lumen, and two or more tendons slideably situated in the wall. A transition portion comprises two or more actuation rods, each being mechanically coupled to one of the two or more tendons, two or more springs, and two or more distal stoppers, wherein the transition portion is configured such that movement of each distal stopper causes movement of each tendon. Proximal to the transition portion is a conduit portion comprising two or more lumen filled with air or hydraulic fluid and each being sealed at the distal end by one of the two or more distal stoppers and a translation portion comprising at least one proximal stopper. A power unit is proximal to the translation portion and is configured to cause hydraulic fluid or air to move through the two or more lumens extending through the conduit such that the tendons move.

According to other aspects of the present disclosure, one or more additional devices, systems, and methods using the medical apparatus are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1 is an exemplary bendable medical apparatus.

FIG. 2(A) illustrates the distal portion of the bendable medical apparatus of FIG. 1.

FIG. 2(B) describes a variation with a longer transition portion.

FIG. 3(A) is an exemplary conduit with center channel.

FIG. 3(B) shows a typical biopsy tool inserted through the center channel.

FIGS. 4(A)-4(E) show a fluid channel cross section of some embodiments of the invention. FIG. 4(A) provides an overview of the bendable medical apparatus. FIG. 4(B) illustrate the cross section at the position marked “B” in the direction indicated. FIGS. 4(C)-4(E) are similar cross sections of FIG. 4(A).

FIG. 5 is an embodiment showing the portion of the system including the power unit and adapter.

FIG. 6 is a simplified drawing for the movement of the articulation tendons.

FIG. 7 shows an embodiment having a single supply line.

FIG. 8 shows a braided insert that can be used in an embodiment of the invention.

FIG. 9 shows an embodiment having a varying diameter fluid/air conduit.

### DETAILED DESCRIPTION OF THE PRESENT DISCLOSURE

One or more devices/apparatuses, systems, and methods for catheters and endoscopes having articulated movement are described.

In some embodiments, there is described a multi-channel lumen conduit for hydraulic fluid or air. In addition to the endoscopes with channels for tools such as biopsy forceps, imaging device, cutter or other surgical tools, there is provided herein additional channel(s) for hydraulic/pneumatic channels within the medical device that control the tendon the provide bendability to the distal end of the medical apparatus. In some embodiments, a combination of return springs and hydraulic (or pneumatic) lumens in a multi-lumen tube are used to actuate the tendons.

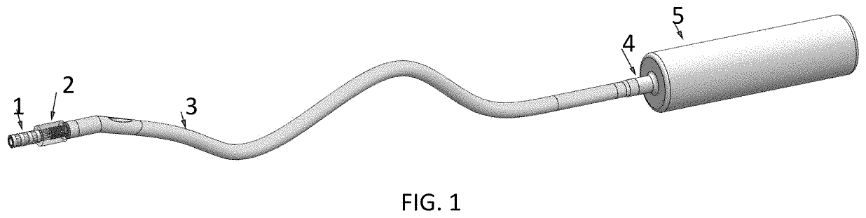

FIG. 1 is a diagram of a bendable medical apparatus describing one embodiment. This system consists of an articulated movement portion 1, a transition portion 2 (which translates changes in hydraulic/pneumatic pressure to movement), a fluid/air conduit portion 3, a translation portion 4 (which translates movement to changes in hydraulic/pneumatic pressure), and a power unit 5 (which contains a motor and a mechanism to translate motor movement to linear movement). Thus, in use, the articulated movement portion 1 is articulated by pneumatic or hydraulic forces activated by the power unit 5.

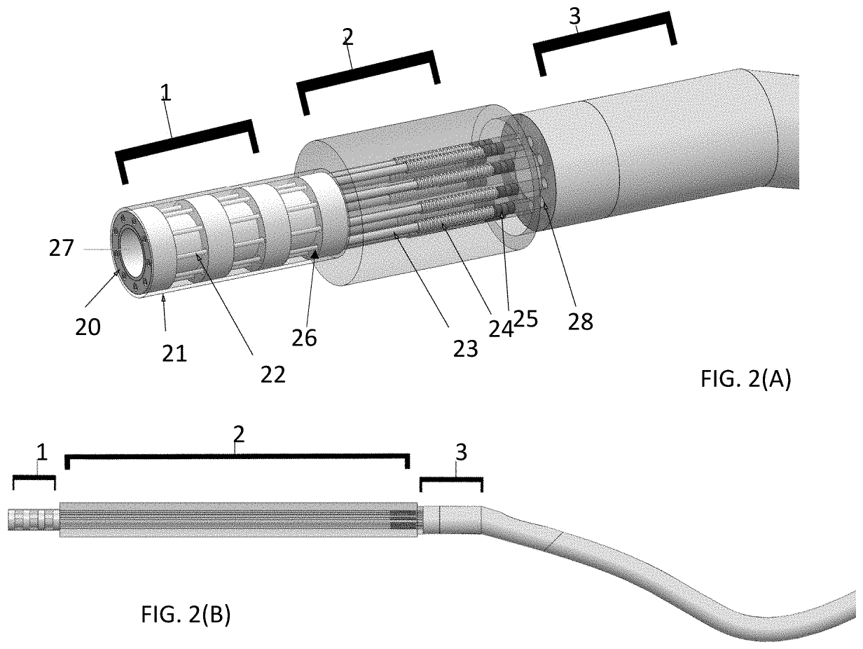

FIG. 2(A) depicts a close up of the exemplary device of FIG. 1 distal to the power unit. In this embodiment, the articulated movement portion 1, is comprised of four cylindrical guidance rings 20, creating a central lumen 27 extending through the device. Extending within these cylindrical rings 20 are a plurality of lumens 26 which contain a plurality of articulation tendons 22. The tendons are anchored within the most distal cylindrical guidance rings.

An outer sheath 21 may be formed around the articulated movement portion, and it may also extend to cover the transition portion 2. The transition portion 2 is comprised of actuation rods 23 which are attached to the articulation tendons 22. The transition portion also includes return springs 24, and distal stoppers 25, where the return springs 24 will return the actuation rods 23 to their distal position when not acted on by movement of the distal stopper 25. The transition portion may be provided as shown in FIG. 2(A) where the return springs 24 are proximal to the actuation rods 23 and the distal stoppers 25 are proximal to the return springs 24. Other embodiments provide for a different configuration where the return springs are proximal to the return springs 24. In this embodiment, the return springs are inside of the conduit portion 3. In the embodiment shown in FIG. 2(A), the fluid/air conduit 3 contains multiple lumens 28 which create individual channels that can be used to activate the articulation tendon. In some embodiments, the articulation tendons 22 have a smaller dimeter than the actuation rods 23, making the actuation rods easier to grip and the tendons more flexible. They may be welded together or may be formed integrally. Particularly where additional gripping is not required, they could have the same diameter. The distal stopper 25 may be a portion at the end of the actuation rod 25 that is greater in diameter or may be, for example, an O-ring

The articulated movement portion 1, is shown with four cylindrical rings in FIG. 2(A). However, this portion may take many other forms. For example, it can have multiple separate bendable sections each having one or more cylindrical rings and two or more tendons attached to one of the cylindrical rings. Alternatively, the articulated movement portion 1 may contain a solid wall extending the length of the portion with no separations to create rings; it may contain a solid wall material but with cut outs to facilitate bendability. In yet other embodiments, the articulated movement portion may contain both cylindrical rings at the distal end and a solid wall located more proximally. While the cylindrical rings are an effective way to allow for a high bending radius, other methods will work as well, or the bendable medical apparatus may not need to produce as sharp of a turn radius. The tendons should be anchored to the solid wall at a distal portion of the wall. In embodiments having more than one bendable section within the articulated movement portion 1, some tendons are anchored at the distal portion of each section. Other embodiments that may work with the bendable medical apparatus are described in U.S. Pat. Nos. 9,144,370; 10,687,694; U.S. Pat. Pub. 2018/0296, 800; U.S. Pat. Pub. 2018/0243, 900; U.S. Pat. Pub. 2018/0311,006; U.S. Pat. Pub. 19/0105, 468; U.S. Pat. Pub. 2021/0121,162; WO2018/204202; WO/2020/086749; WO/2020/092096; and WO/2020/092097, each of which are herein incorporated by reference in their entirety.

In some embodiments, the return springs 24, at neutral pressure, return the tendon to the neutral position that can release the push' from the actuated actuation rods 23. In other embodiments, the return springs 24 are preloaded with an applied pressure. When this specific pressure is applied, the tendon (and the articulation movement portion for application to all tendons) is at a neutral position. When the pressure is fully relived for a spring, the spring will return to its natural length and ‘pull’ the tendon to a maximum amount.

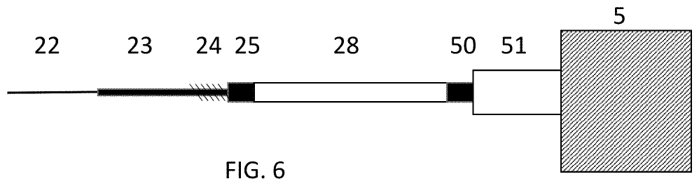

In some embodiments, the connection between the conduit and the distal stopper is not linear such as is as shown in FIG. 6. In these embodiment, the two or more lumen filled with air or hydraulic fluid interact with the distal stopper on the distal side of the distal stopper instead of on the proximal side of the distal stopper. The lumen will run parallel to the spring and distal stopper until a part of the transition portion distal to the movement of the stopper where a fluid or hydraulic connection links the two.

In some additional embodiments, the transition portion comprises two springs for every actuation rod and stopper. The springs are located on both sides of the stopper and act to return the stopper to the neutral position (or beyond the neutral position) at equilibrium. The presence of both springs acts as a safety mechanism, in case there is a leak in the pressure line. To have the ability to push as well as pull in these embodiments, there are hydraulic/pneumatic chamber on both sides of the stopper that are actuated independently.

In yet other embodiments, in the transition portion, the stopper is attached to a pinion. The tendon is then attached to a rack, which is in contact with the pinion. As the stopper is moved by hydraulic or pneumatic force, this causes the pinion to rotate, thereby moving the rack linearly. Because the tendon is attached to the rack, when the rack moves, the tendon moves as well.

FIG. 2(B) depicts an elongated transition portion 2 as compared to FIG. 2(A) . . . . In this embodiment, the bendable medical apparatus is particularly useful in a location where the bendable properties are needed at a location away from the entry point. For example, this embodiment could be used in the lung, where a long transition portion 2 can be inserted into the patient's trachea and optionally into the airways to provide bendability within the branching lung. Alternatively, any of the articulated movement portion 1, the transition portion, 2, and the fluid/air conduit 3 may be lengthened or shortened dependent upon the specific use of the bendable medical apparatus.

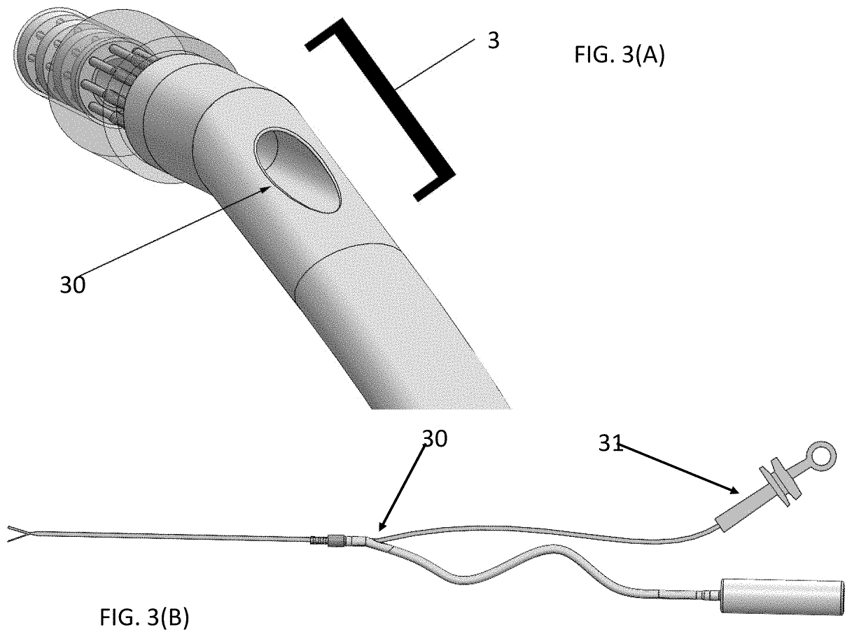

FIG. 3 is a close-up of the central lumen port 30 in the fluid/air conduit portion 3. The central lumen port 30 allows a medical tool 31 such as a biopsy tool, a camera, a cutting device, etc. to be inserted into the articulated movement portion without disturbing the hydraulic/pneumatic channels. FIG. 3(B) shows a typical biopsy tool 31 inserted through the central lumen port 30.

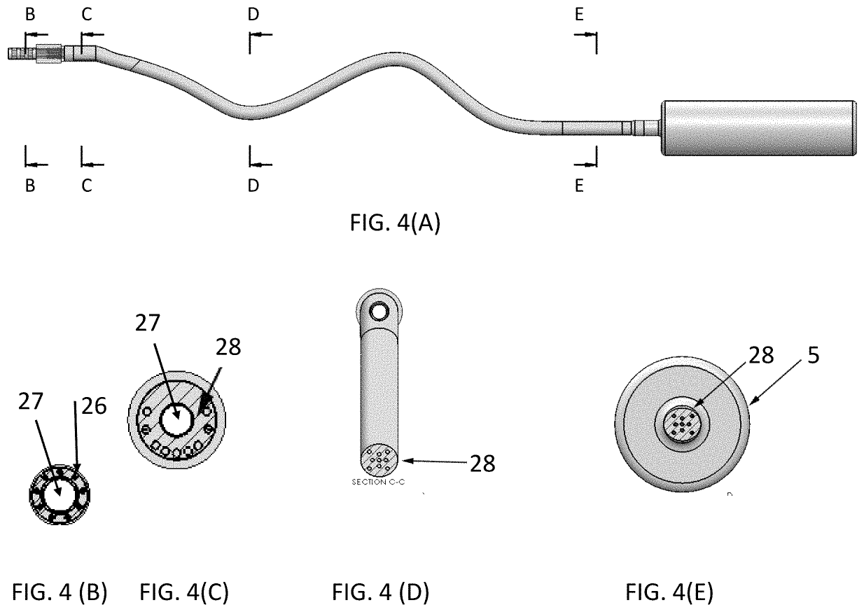

FIGS. 4(A)-4(E) further illustrates an exemplary embodiment having fluid/air channels at various positions throughout the device. Cross section FIG. 4(B) shows at the articulated movement portion having multiple lumens 26 and an open center channel 27 for biopsy and other tools to emerge from the tip. Also the channels 26 for the articulation tendons are evenly distributed around the perimeter of the device.

Cross section FIG. 4(C) shows the channels 28 which feed each individual articulation tendon 22 are now biased toward one side, allowing the center channel 27 to exit the conduit at the central lumen port 30 without piercing any of the hydraulic/pneumatic channels 28. Cross section of FIG. 4(D) is located within the fluid/air conduit portion 3 and shows the individual hydraulic/pneumatic channels 28 distributed after any tool and the center channel 27 has exited the conduit. Cross section FIG. 4(E) shows the individual hydraulic/pneumatic channels 28 as they enter the power unit 5.

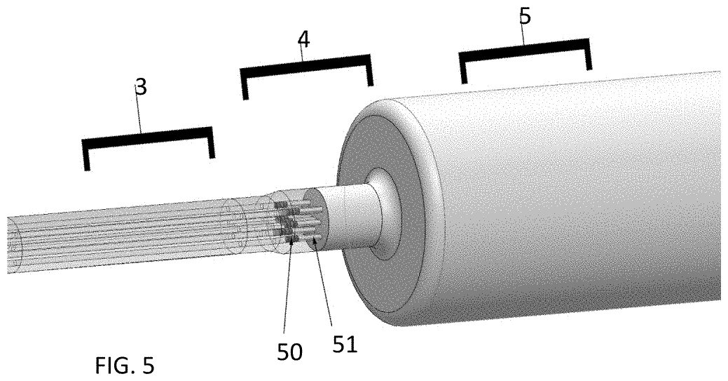

FIG. 5 depicts the translation portion 4 for the hydraulic/pneumatic conduit 3, leads to the translation portion 4. Contained in this adapter are proximal stoppers 50, attached to actuation members 51. The actuation members 51 are, in turn, acted upon by the power unit 5. In some embodiments, the actuation member 51 is a connection unit, such as a rod or a wire between the power unit 5 and the proximal stopper 50. In some embodiments the power unit 5 individually actuates each proximal stopper 50. In other embodiments, the actuation of the individual stoppers is provided by a distributor. In yet other embodiments, one power unit 5 is used for each actuation member 51.

In use, the system as described by this embodiment functions as follows and as illustrated by FIG. 6: the power unit 5 is instructed to activate one (or several, but only a single is shown in FIG. 6 for simplicity) of the actuation members 51. This member is attached to a proximal stopper 18, which therefore increases or relieves the pressure of the fluid or air in the channels 28 located in the conduit 3. If the pressure increases, it causes the associated proximal stopper 25 in the transition portion 2 to move further proximal to the motor 5, causing a movement in the associated articulation tendon 22. As soon as the power unit 5 relieves pressure on this line, the return spring 24, pushes the proximal stopper 25 back to a neutral position, pulling the articulation tendon with it. This push/pull movement can be repeated and can be simultaneous with similar movements in the other channels.

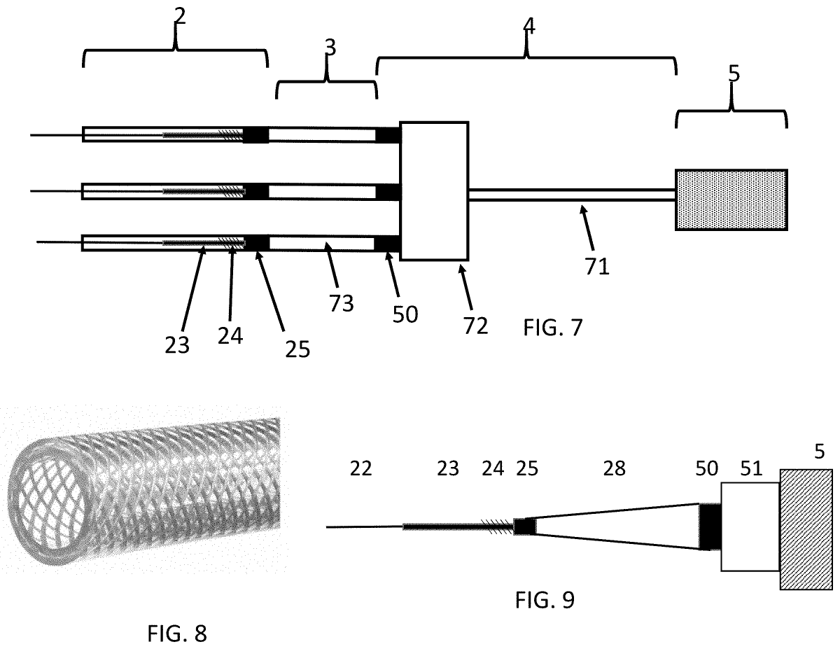

FIG. 7 describes an alternate embodiment in which articulated movement portion 1 is enabled by a master hydraulic/pneumatic line 71. The hydraulic/pneumatic fluid flowing into the channels 71 are controlled by a distributor 72. This distributor 72 controls the flow in to a plurality of different lumens 73 such that the correct lumens 73 receive the needed fluid/air in order to produce the desired tip articulation. The distributor 72 is in turn supplied by a master hydraulic/pneumatic line 71 which has fewer lumen channels than the number of lumens 73. In some embodiments, a plurality of solenoids are provided in each of the transition portions.

The fluid/air conduit portion 3 is preferably flexible such that it can be manipulated with minimal trauma to the patient. However, because of the nature of this invention, the lumens which the gas or fluid travels through needs to be as non-compliant as possible. One solution is to have braided inserts in the gas/fluid lumens 28, such as shown in FIG. 8. Such a structure allows bending, but not radial expansion, allowing the gas/fluid to move the stoppers in this concept. Other means of creating the fluid/air conduit with sufficient flexibility and noncompliance include selecting a material or composite material for the proper balance between stiffness and flexibility. In some preferred embodiments this flexibility provides for the articulated movement portion of the medical apparatus to bend more than 90°, or preferably more than 135° or up to 180° while having a radius of 10 cm or more preferably 6 cm. There are also preferred embodiments where the articulated movement portion is non-compliant such that that the diameter of the lumen extending through the conduit portion will not increase more than 10%, or more preferably not more than 5% or even more preferably not more than 2%.

In another embodiment, there is provide a variation to the fluid/air conduit portion 3 as shown in FIG. 9 The inner diameter of the channels 28 are of varying inner diameter—a smaller diameter at the end distal to the power unit 5 FIG. 1 as compared to the end proximal to the power unit 5 FIG. 1. This embodiment creates a system having a particular advantage where small movements of the proximal stoppers 50 as shown in FIG. 5 produce a proportionally larger movement at the distal stoppers 25 as shown in FIG. 2. The variation may be as shown-consistently decreasing in diameter. Alternatively, it may be step-wise or changing over only a portion of the fluid/air conduit 3. This embodiment can be implemented with the other embodiments as described herein.

In another embodiment, there is described a system where the return springs 24 in FIG. 2 are absent. The movement of the actuation rods 23 in FIG. 2 are dictated by increasing pressure in lumen 28 to push the distal stopper 25 or suction in lumen 28 to pull the distal stopper 25.

In some embodiments, the movement of an articulated endoscope device could require multiple tendons to be pulled and pushed. For example, it could require 2, 3, 4, 5, 6, 7, 8 or more tendons. These tendons can have both push and pull motion. Further, the articulated endoscope may comprise a single bendable section with, for example, 1, 2 or 3 tendons. In other embodiments, the endoscope may have 2, 3, 4, 5 or more separate bendable sections, each controlled by its own 1, 2, or 3 (or more) tendons. An exemplary multiple section endoscope with these features is described in U.S. Pat. Pub. 2019/0105468 and WO/2020/086749, each of which are herein incorporated by reference in their entirety. These separate bendable sections may each be the same length or different.

These tendons and the endoscope itself are flexible and able to navigate, for example, various lumens within a body. In some embodiments, the navigation occurs near the entrance to the body, but in others, a long section or support structure will be used to get the articulating endoscope device to the area in interest, such as using a bronchoscope to enter and navigate the trachea. This long length can affect how the endoscope is preferably handled and controlled.

In some embodiments, the volume and weight of the drive motors/mechanisms could be ergonomically undesirable to contain in the handle. Thus, in one embodiment, the drive mechanism is housed in an enclosure, separate from the handle.

To simply lengthen the tendons could cause, in some embodiments, complications of increased actuation forces and/or tendon kinking. One solution to this is to use a combination of return springs and hydraulic (or pneumatic) in a multi-lumen tube to actuate the tendons.

As described herein there may be multiple hydraulic actuators coupled through a multi-lumen conduit. These multiple actuators can control the same bendable section or different bendable sections within an endoscope.

Thus, there is provided an endoscopic device comprising: hydraulic (or pneumatic) actuators to pull and push control tendons in an articulated tip endoscopic device; a power unit, remote from the articulated tip, that is configured to apply forces on one or more actuation rod(s) with stopper(s) on the end. The stoppers are preferably placed in the one or more fluid/air filled channel(s) of a conduit. As the stoppers move, the compression of the air/fluid causes movement in a stopper(s) at the far end of the channel.

This distal stopper, in turn, causes an actuation tendon(s) to move toward the tip. This tendon movement causes the tip to bend. When power unit of the system relaxes the pressure on the fluid/air, then return spring in the tip push the distal stopper(s) back to a neutral position, bending the tip back to a neutral position.

In some embodiments, multiple lumen fluid/air channels are provided with multiple simultaneous actuation movements.

Although the disclosure herein has been described with reference to particular embodiments, it is to be understood that these embodiments are merely illustrative of the principles and applications of the present disclosure (and are not limited thereto). It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications, equivalent structures and functions.

It is to be understood that the present disclosure is not limited to any particular embodiment. It is also to be understood that the terminology used herein is for the purpose of describing exemplary embodiments only, and is not intended to be limiting.

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope and spirit of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one (or two) or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

1. A medical apparatus comprising:
an articulated movement portion comprising:
a central lumen extending the length of the articulated movement portion,
a wall formed about the central lumen, and
two or more tendons slideably situated in the wall;
a transition portion comprising:
two or more actuation rods, each being mechanically coupled to one of the two or more tendons,
two or more springs, and
two or more distal stoppers,
wherein the transition portion is configured such that movement of each distal stopper causes movement of each tendon;
a conduit portion comprising two or more lumen filled with air or hydraulic fluid and each being sealed at the distal end by one of the two or more distal stoppers;
a translation portion comprising two or more proximal stoppers; and
a power unit proximal to the translation portion that is configured to cause hydraulic fluid or air to move through the two or more lumens extending through the conduit such that the tendons move.

2. The medical apparatus of claim 1, wherein the spring is configured such that when the power unit releases the pressure on the hydraulic fluid/air, then return spring move the distal stopper(s) to a neutral position or beyond, thereby bending a tendon to a neutral position.

3. The medical apparatus of claim 1, wherein at least a portion of wall formed about the central lumen comprises an inner sheath and a plurality of cylindrical rings where the two or more tendons are anchored to a distal cylindrical ring and slideably situated in the remaining plurality of cylindrical rings.

4. The medical apparatus of claim 1, wherein the two or more actuation rods have a greater diameter than the two or more tendons.

5. The medical apparatus of claim 1, wherein, in the transition portion,
the two or more springs are proximal to the two or more actuation rods, and
the two or more distal stoppers are proximal to the two or more springs.

6. The medical apparatus of claim 1, wherein, in the transition portion,
the two or more distal stoppers are proximal to the two or more actuation rods, and
the two or more springs are proximal to the two or more stoppers.

7. The medical apparatus of claim 1, wherein the conduit portion is formed from a material or combination of materials that is flexible enough to bend more than 90 degrees with a radius of 10 cm and is non-compliant such that that the diameter of the lumen extending through the conduit portion will not increase more than 10%.

8. The medical apparatus of claim 7, wherein the conduit portion is formed from a material or combination of materials that is flexible enough to bend more than 135 degrees with a radius of 6 cm and is non-compliant such that that the diameter of the lumen extending through the conduit portion will not increase more than 5%.

9. The medical apparatus of claim 1, wherein the articulated movement portion comprises at least two bending sections, each configured to be bent by at least two tendons.

10. The medical apparatus of claim 9, wherein each bending section comprising at least three tendons anchored to the distal end of the bending section.

11. The medical apparatus of claim 9, wherein the articulated movement portion comprises at least three bending sections, each configured to be bent by three tendons.

12. The medical apparatus of claim 11, wherein the medical apparatus also comprises a total of six or more of each of: actuation rods, springs, distal stoppers, lumens, and proximal stoppers.

13. The medical apparatus of claim 1, wherein the translation portion further comprises a master line filled with air or hydraulic fluid and a distributer that is configured to control the flow of air or hydraulic fluid in the two or more lumens.

14. The medical apparatus of claim 13, further comprising two or more solenoids in the transition portion.

15. The medical apparatus of claim 1, wherein the medical apparatus is sized and configured for use in the lungs.

16. A medical apparatus comprising:
an articulated movement portion comprising:
a central lumen extending the length of the articulated movement portion, a wall formed about the central lumen, and at least nine tendons slideably situated in the wall;
a transition portion comprising:
at least nine actuation rods, each being mechanically coupled to the at least nine tendons, at least nine springs, and at least nine distal stoppers, wherein the transition portion is configured such that movement of each distal stopper causes movement of each tendon;
a conduit portion comprising at least nine lumen filled with air or hydraulic fluid and each being sealed at the distal end by the at least nine distal stoppers;
a translation portion comprising at least nine proximal stopper; and
a power unit proximal to the translation portion.

17. A medical system comprising:
a medical apparatus comprising:
an articulated movement portion comprising:
a central lumen extending the length of the articulated movement portion,
a wall formed about the central lumen, and
two or more tendons slideably situated in the wall;
a transition portion comprising:
two or more actuation rods, each being mechanically coupled to one of the two or more tendons,
two or more springs, and
two or more distal stoppers,
wherein the transition portion is configured such that movement of each distal stopper causes movement of each tendon;
a conduit portion comprising two or more lumen filled with air or hydraulic fluid and each being sealed at the distal end by one of the two or more distal stoppers;
a translation portion comprising at least one proximal stopper; and
a power unit proximal to the translation portion that is configured to cause hydraulic fluid or air to move through the two or more lumens extending through the conduit such that the tendons move;
a tool configured to fit within the central lumen; and
a computer configured to control the power unit.

18. The medical system of claim 16, wherein the tool is a camera or a biopsy tool.
